(19) United States
(12) Patent Application Publication
(10) Pub. No.: US 2006/0241383 A1
(43) Pub. Date: Oct. 26, 2006

(54) Method of operating a medical imaging system

(75) Inventors: Estelle Camus, Erlangen (DE); Martin Kleen, Furth (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/391,985

(22) Filed: Mar. 29, 2006

(30) Foreign Application Priority Data
Mar. 30, 2005 (DE) 10 2005 014 445.4

(51) Int. Cl. A61B 5/05

(52) U.S. Cl. 600/413

(57) ABSTRACT

A trigger device feeds to a control device for a medical imaging system a number of trigger pulses which all correspond to a predetermined phase angle of an object, e.g. of a heart which is the same for all trigger pulses. The control device activates an image recording device at least on the basis of a few of the trigger pulses so that the image recording device records a sequence of images of the object in each case, as from a start time relative to the initiating trigger pulse and ends the recording of the sequence at a stop time, so that the images (Bk) of this sequence have predetermined time offsets to the initiating trigger pulse. The control device determines the stop time such that it lies before a start time which is determined on the basis of the trigger pulse immediately following the initiating trigger pulse. The image recording device feeds the recorded sequences to the control device which stores the sequences fed to it.

### CROSS REFERENCE TO RELATED APPLICATIONS

This application claims priority to the German Application No. 10 2005 014 445.4, filed Mar. 30, 2005 which is incorporated by reference herein in its entirety.

### FIELD OF INVENTION

The present invention relates to a method of operating a medical imaging system which features a control device, an image recording device and a trigger device.

### BACKGROUND OF INVENTION

These types of methods of operation, the corresponding data medium and also the medical imaging systems as such are generally known. For example angiography systems are known which feature the above-mentioned components and for which methods of operation are generally known. Such systems are used among other things for conducting examinations of the human heart.

It is thus known for example that a number of trigger pulses, which all correspond to predetermined phase angle of an object which is the same for all trigger pulses, are fed sequentially by the trigger system to the control device. On the basis of the trigger pulses the imaging system records an image which has a predetermined time offset to the trigger pulse in each case. The image recording system feeds the recorded images to the control device which stores the images fed to it. Since they all have the same time offset to the trigger pulse, the images originate as a rule from approximately the same phases of the object. They are thus assembled into an image group by the control device and processed as a group of images. The processing in this case consists of what is known as a DSA (DSA=Digital Subtraction Angiography) or also of a simple sequential order of the images.

Since its introduction, the Digital Subtraction Angiography mentioned has found numerous applications in radiology. The basic idea in this case is the subtraction of an x-ray image, which was taken before an injection of contrast means, from a second x-ray image taken after the injection of the contrast means. The differential image thus determined shows the distribution of the contrast means in the image and thereby in the organ significantly more clearly and more plainly than the original image.

### SUMMARY OF INVENTION

An important prerequisite for digital subtraction angiography consists of the recorded object being at the identical position for the two images which are to be subtracted from each other and also the other imaging parameters remaining the same. The position in such cases includes both the location and also the orientation of the object. For moving objects (for example the beating heart of a person) it is thus not simply permissible to record two images (one with, one without contrast means) and to subtract the two images from one another. Instead it must be ensured that the object is located in the same position. To guarantee this the recordings are triggered by means of an ECG for example.

Furthermore it is known for example that pulses which correspond to a predetermined phase position of an object are fed to the control device. The control device controls the image recording device independently of the pulses in such a way that the latter records a sequence of images of the object as from a start time and ends the recording of the sequence at a stop time. In this case the images of this sequence are offset in time in relation to the pulses. The image recording device feeds the recorded sequence to the control device, which stores the sequence fed to it as well as the pulses.

The recording of a sequence of images is used for example to trace the timing sequence of the entry and the flushing out of a contrast means from the human heart. To this end the injection of the contrast means is connected with the recording of the sequence. The recording of the sequence is for example ended if the contrast means has reached a specific point of the blood vessel to be checked or is flushed out of the blood vessel again. The sequence thus extends as a rule over a number of pulses or—in the case of cardio-angiography—over a number of heart cycles. The images of the sequence are recorded in this case at a fixed, high image rate of e.g. 30 images per second.

In many cases the change in the contrast in the recorded images is so small, that an evaluation of the sequence is no longer possible or is only possible with considerable difficulty. Differential images are thus also often determined in the recording of image sequences. Groups of images are recorded for this purpose. For each image group an image which lies in the phase range of the object which is specific for this group of images is determined from each cycle of the object. A cycle in this case corresponds to the period from pulse to pulse. Since the phase angles of the images within the image groups however do not match exactly, falsifications and artefacts occur.

An object of the present invention is to create an improved method of operation and avoiding the mentioned disadvantages.

The object is achieved for a method of operation for a medical imaging system, which features a control device, an image recording device and a trigger device by
the trigger device sequentially feeding to the control device a number of trigger pulses, which all correspond to a predetermined phase angle of an object which is the same for all trigger pulses.
by the control device activating the image recording device at least as a result of a number of the trigger pulses such that the image recording device in each case records a sequence of images of the object starting from a start time relative to the initiating trigger pulse and ends the recording of the sequence at a stop time, so that the images of this sequence exhibit predetermined time offsets to the initiating trigger pulse,
by the control device den defining the stop time such that it lies before a start time which is determined on the basis of the trigger pulse immediately following the initiating trigger pulse, and
by the image recording device feeding the recorded sequences to the control device and the control device storing the sequences fed to it.

The object is further achieved by a computer program for a control device of a medical imaging system, by means of which such a method of operation is able to be executed The data medium can in this case for example be a removable medium which can be brought into effective connection with the control device at any time via a corresponding interface. Example of such removable media are a CD-ROM or a USB memory stick. Alternately the data medium can however also be embodied as a memory device permanently assigned to the control device, for example as a hard disk or as a semiconductor memory.

Based on the inventive procedure it is for example possible for the control device to assemble the images of the sequences which feature the same time offset to the initiating trigger pulse in each case into image groups, and to processes them in image groups. It is especially possible, within the image groups, to determine differential images and to further evaluate these differential images. In this case for example the difference from a particular image of the image group—e.g. of the first or the last chronological image of this image group—can be determined. Alternatively it is also possible to determine the differences of images immediately following one another in time or within the group. The further processing can however also be undertaken by an evaluation processor separate from the control device.

As a rule the trigger device records an actual phase angle of the object and then emits a trigger pulse when the actual phase angle corresponds to a reference phase angle. For the typical case in which the object is the beating heart there is for example an ECG triggering. In an individual case it can however also be worthwhile for stimulation pulses to be specified for the object by a stimulation device and for the trigger device to derive the trigger pulses from stimulation pulses. If for example the heart is beating very irregularly a heart pacemaker can be used as a stimulation device of which the output signal is fed on the one hand to the heart and on the other hand to the trigger device. Furthermore a corresponding stimulation of the object is also possible without deriving the trigger pulses from stimulation pulses.

As the rule directly consecutive images within each sequence are equidistantly spaced in time. For example 30 images can be recorded per second. It is however also possible that within each sequence directly consecutive images are not equidistantly spaced in time. The latter can be particularly useful if a number of images are to be recorded per heartbeat during the diastole phase.

The image recording device needs a predetermined time for the correct recording of an image. If the stop time of a sequence thus lies immediately before the start time of the subsequent sequence this can under some circumstances cause problems for the correct recording of the images. There are a number of options for resolving or working around such problems.

Thus it is possible for example for a trigger pulse only to initiate the recording of a sequence if, between recording of the last image of a previous sequence and this trigger pulse there is a period of time which exceeds a minimum wait time. This minimum wait time is in this case of course selected so that it is at least as great as the time needed by the image recording device for the correct recording of an image.

Alternatively it is also possible for a trigger pulse to always initiate the recording of a sequence. In this case for example the image recording device can overwrite the last image of the previous sequence before feeding it to the control device. This last image is then thus not fed to the control device at all.

Alternatively it is also possible for the image device not to feed the first image of the corresponding image sequence to the control device. In this case the first image of the corresponding sequence is thus not fed to the control device.

In another alternative it is also possible that although the first image of the initiated sequence is fed to the control device, is not however evaluated by a control device.

In the cases described above the stop time of a sequence is determined on the basis of the subsequent trigger pulse. This procedure is useful if the time interval between the trigger pulses is not already known in advance. If the time interval is known to the control device however it is also possible for the control device to determine the stop limes on the basis of the time interval and the corresponding start times. In this case the stop time of a sequence is thus determined on the basis of the same trigger pulses on the basis of which the start time of this sequence is determined.

Finally it is also possible for the start time of a sequence to be delayed in relation to the trigger pulse to be initiated by a delay time which is at least as great as the minimum wait time. The recording of the last image of a sequence can then be correctly concluded before the first image of the new sequence is recorded.

As a result of pulse changes during the recording of the sequences it can occur that the number of images varies from sequence to sequence. Preferably the control device thus determines for each sequence the number of images contained in the relevant sequence as well as their minimum or maximum. If the control device determines the minimum it does not evaluate the images recorded last for the sequences which have a larger number of images than the minimum, where such sequences exceed the minimum. If the control device determines the maximum it duplicates the last image recorded for the sequences containing a smaller number of images than the maximum until these sequences to contain the maximum number of images. This duplication is possible as a rule because the last images recorded generally lie in the diastole phase of the heart No changes or only slight, tolerable changes thus occur in these images.

### BRIEF DESCRIPTION OF THE DRAWINGS

Further advantages and details emerge from the subsequent description of an exemplary embodiment in conjunction with the drawings. The Figures show the following basic diagrams:

FIGS. 1 and 2 a medical image recording system in each case,

FIGS. 3 and 4 a timing diagram in each case,

FIG. 5 a number of image sequences and the grouping of images,

FIG. 6 a variant of FIG. 4

FIG. 7 to 9 flowcharts

FIG. 10 a timing diagram and

FIGS. 11 and 12 variants of FIG. 5

### DETAILED DESCRIPTION OF INVENTION

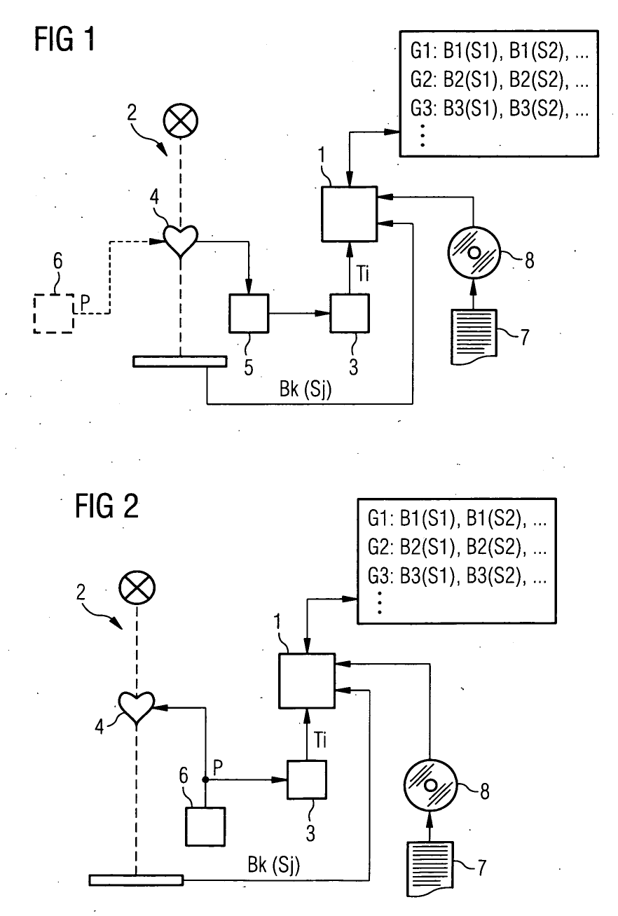

In accordance with FIGS. 1 and 2 a medical imaging system is typically embodied as an angiography system. It could however also be embodied as another medical imaging system, e.g. as a computer tomograph, or as an ultrasound imaging system. The medical imaging system features at least one control device 1, at least one image recording device 2 and a trigger device 3.

The control device 1 controls the mode of operation of the medical imaging system, especially of the image recording device 2. The image recording device 2 records images of an object 4, e.g. of the heart 4 of a person. The trigger device 3 records phase angles of the object 4. It feeds a number of trigger pulses Ti (I=1, 2, 3 . . . ) sequentially to the control device 1. The trigger pulses Ti all correspond to a predefined phase angle o f the object, e.g. the beginning of the systole phase of the heart. The predefined phase angle is thus the same for all the trigger pulses Ti.

In accordance with FIG. 1 the trigger device 3 is connected to a sensor device 5, so that it is able to record the actual phase angle of the object 4. In this case the trigger device 3 compares the actual phase angle of the object 4 recorded with a reference phase angle, e.g. the beginning of the systole phase of the heart whenever a match is produced, the trigger device 3 issues a trigger pulse Ti.

If necessary a stimulation device 6 can be present, e.g. a heart pacemaker. The stimulation device 6 in this case specifies stimulation pulses P to the object. 4 This is shown as a dashed line in FIG. 1.

In accordance with FIG. 2 the stimulation pulses P are also fed to the trigger device 3. This then derives trigger pulses Ti from the stimulation pulses P. Otherwise the embodiment of FIG. 2 corresponds to that of FIG. 1.

The control device 1 controls the medical imaging system on the basis of a control program 7, which is stored on a data medium 8. On the basis of the control program 7 the control device 1 controls devices such as the image recording device 2 based on at least some of the trigger pulses Ti. These trigger pulses Ti which initiate a subsequent activation of the image recording device 2 are referred to below as initiating trigger pulses Ti.

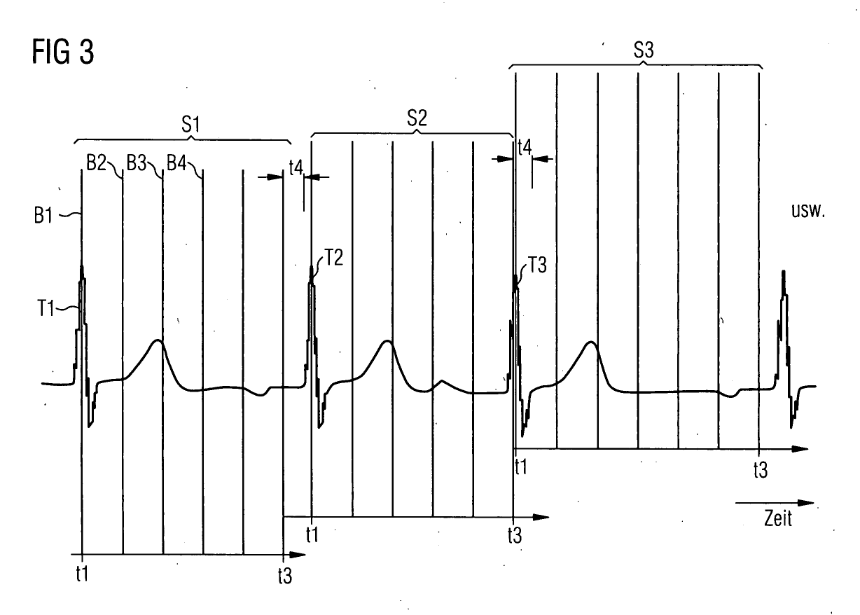

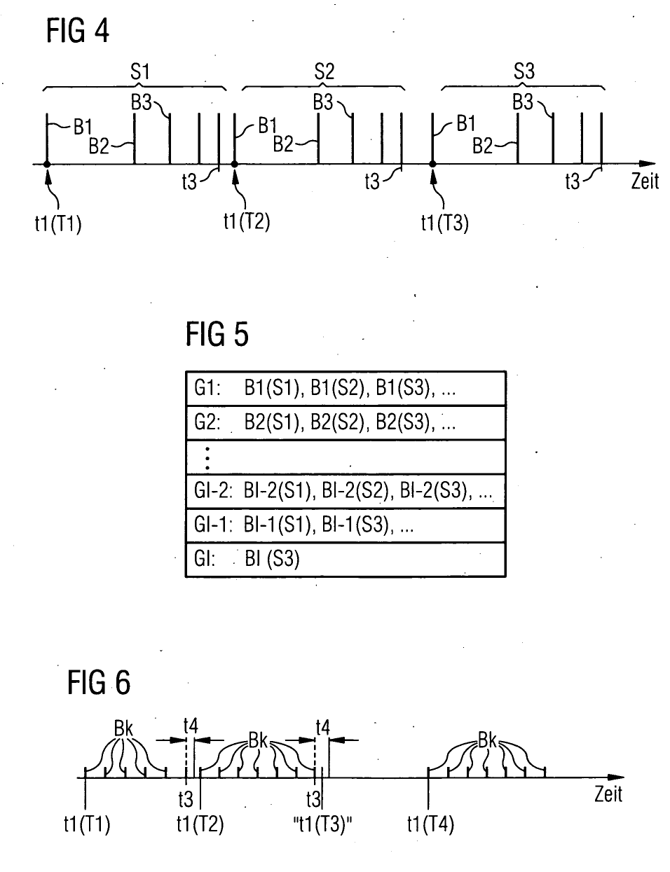

The image recording device 2 is activated in accordance with FIGS. 3 and 4 in such a way that the image recording device 2, starting at a start time t1 in each case, records a sequence Sj (j=1, 2, 3 . . . ) of images Bk (k=1, 2, 3 . . . ) of the object 4. The start times t1 in this case are either identical to the times at which the initiating trigger pulses Ti occur or exhibit a prespecified delay time t2 (see FIG. 10) in relation to these times.

The individual images Bk of a sequence Sj are recorded in each case at specific times after the relevant start time t1. The images Bk of the relevant sequence Sj thus have a prespecified time offsets to the initiating trigger pulse Ti. Directly consecutive images Bk of a sequence Sj can in this case be spaced equidistantly in time from each other in accordance with FIG. 3. The intervals can however also vary, as shown in FIG. 4.

The recording of the sequences Sj is ended in each case when a stop time t3 is reached. The control device 1 determines the stop time t3 of each sequence Sj in such cases so that it lies before a start time t1 which is determined on the basis of the trigger pulse Ti immediately following the initiating trigger pulse Ti. This is discussed in more detail below.

The image recording device 2 feeds the recorded sequences Sj to the control device 1, which stores the sequences Sj. The images are fed and stored individually.

In accordance with FIGS. 1, 2 and 5 the control device 1 groups together the images Bk of the sequences Sj which have the same time offset to the initiating trigger pulse Ti in each case, into image groups GI (1=1, 2, 3, . . . ). The images Bk are also processed in groups. This is also discussed in more detail below.

As can be seen from FIG. 3, the time interval between stop times t3 of a sequence Sj and the occurrence of the subsequent trigger pulse Ti varies. In such cases it is possible as an alternative for this interval to be greater than or less than a minimum wait time t4. The minimum wait time t4 is the time which the image recording device 2 needs to correctly record an image BK and feed it to the control device 1. To avoid conflicts it is thus necessary to define the stop times t3 in a suitable manner or to take other measures which prevent the conflicts.

In the procedures in accordance with FIG. 6 to 8 for example the stop time t3 is kept indeterminate until a new trigger pulse Ti occurs. Then the start time t1 is determined for this new trigger pulse Ti and with reference to this start time t1 the stop time t3 of the previous sequence is set to the time of the recording of the last image Bk recorded.

In accordance with FIG. 6, after each recording of an image Bk the system first waits for the minimum time t4. If a trigger pulse Ti now occurs before the minimum wait time t4 has elapsed, this trigger pulse Ti does not initiate the recording of a sequence Sj of images Bk. Thus, although this trigger pulse Ti causes the ending of the previous sequence Sj, it does not cause the start of a new sequence Sj.

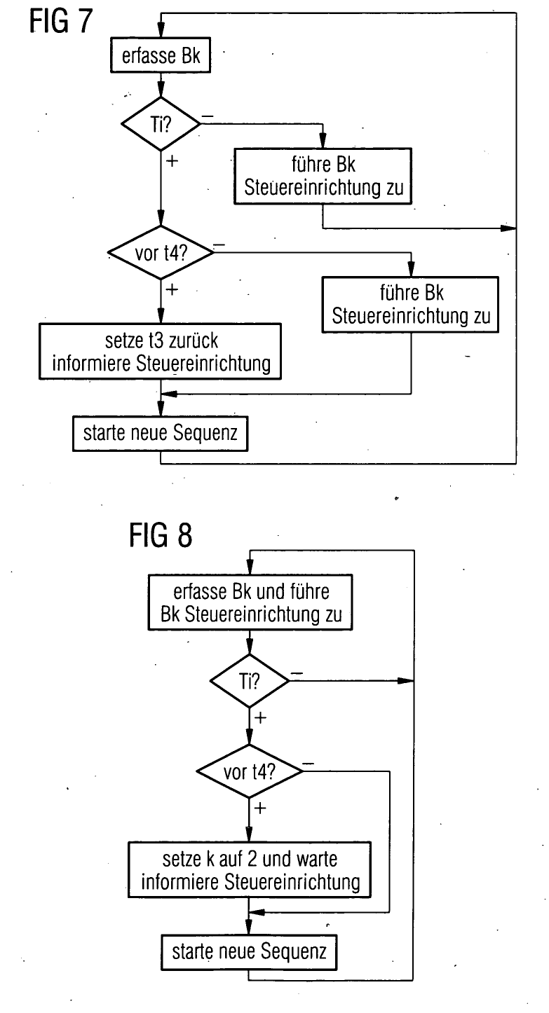

Although the procedure in accordance with FIG. 6 is possible, it is not very advantageous. This is because it can occur that no sequences Sj of individual heart cycles are recorded. It is thus preferable in accordance with FIGS. 7, 8 and 9 for a trigger pulse Ti to initiate the recording of a sequence Sj of images Bk regardless of whether it falls within the minimum wait time t4 or not. The case in which the trigger pulse Ti does not fall within the minimum wait time t4 is not critical in this case and is thus not dealt with in any greater detail below.

If by contrast the trigger pulse Ti falls within the minimum wait time t4, the image recording device 2 can, in accordance with FIG. 7 for example, overwrite the last image Bk of the previous sequence Sj, without previously feeding it to the control device 1. The stop time t3 is postponed in this case by one image Bk and a corresponding indication is transferred to the control device 1.

Alternatively it is also possible in accordance with FIG. 8 for the stop time t3 to be retained and for the image recording device 2 to suppress the recording of the first image B1 of the new sequence Sj. In this case the first image B1 of this new sequence Sj is not fed to the control device 1. In this case too a corresponding indication is transferred by the image recording device 2 to the control device 1.

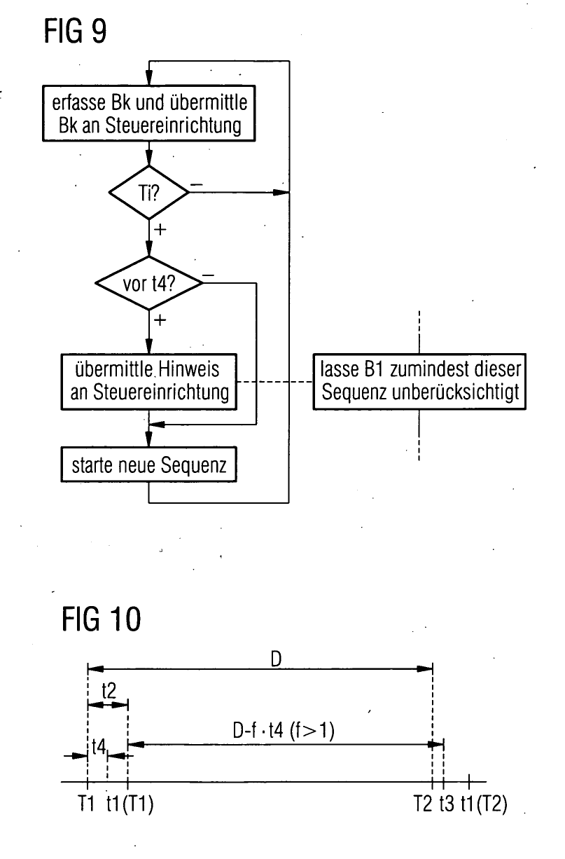

As a further alternative it is also possible in accordance with FIG. 9 for both the last image Bk of the previous sequence Sj and also the first image B1 of the subsequent sequence Sj to be recorded by the image recording device 2 and fed to the control device 1. In this case however a corresponding indication is transferred by the image recording device 2 to the control device 1. The control device 1 is thus in a position to leave the first image B1 of the corresponding sequence Sj—if necessary even all the first images B1 of the sequences Sj—out of its evaluation.

Within the framework of FIG. 6 to 9 the requirement was for a time interval D of the trigger pulses Ti of the control device 1 not to be known in advance and for the start times t1 to coincide with the occurrence of the trigger pulses Ti. In the case of this embodiment the stop times t3 are preferably determined on the basis of the trigger pulse Ti which immediately follows the initiating trigger pulse Ti. If on the other hand the time interval D of the trigger pulses Ti—is known exactly or at least approximately—it is also possible to determine the stop times t3 with reference to the time interval D and the corresponding start times t1. For example it is possible in accordance with FIG. 10 to determine the stop times in accordance with the equation t3
t3=t1+D−ft4

f is in this case a safety factor which must be greater than one. As a rule it will lie between 1.5 and 2.5.

The last procedure described, namely the reliable ending of the recording of a sequence Sj at the right time before the beginning of the recording of a new sequence Sj, is then easily possible in accordance with FIG. 10 if the start times t1 of the sequences Sj are delayed in relation to the initiating trigger pulses Ti by the delay time t2 and the delay time t2 is greater than the minimum wait time t4. This then enables the recording of the currently recorded image Bk of the ongoing sequence Sj to be easily ended correctly on occurrence of a trigger pulse Ti and enables the correct recording of the first image B1 of the subsequent sequence Sj to be started on reaching the next start time t1.

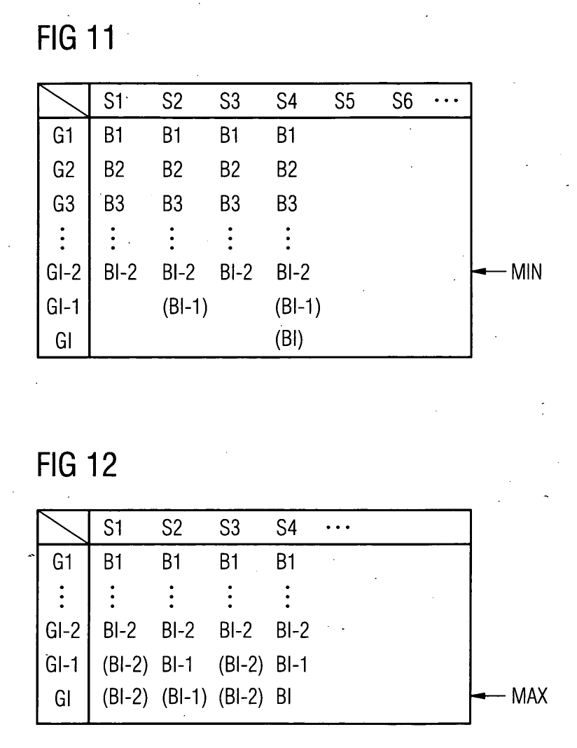

As can be seen from FIGS. 11 and 12, the sequences Sj feature numbers of images Bk. The numbers in these cases, although they are about the same, are not exactly the same Instead they vary by (as a rule) one or two images Bk. The control device 1 thus determines for each sequence Sj the corresponding number of images Bk. In its simplest form thus is done as soon as the images Bk are accepted by the control device 1. The control device 1 then alternatively in accordance with FIG. 11, determines the minimum MIN of the numbers, or in accordance with FIG. 12 the maximum MAX of the numbers.

If the control device 1 in accordance with FIG. 11 determines the minimum MIN, in accordance with FIG. 11 it modifies the evaluation of the sequences Sj containing a larger number of images Bk. This is because the control device 1 does not evaluate the last recorded images Bk of these sequences Sj if they exceed the minimum MIN. This is indicated in FIG. 11 by the fact that these images Bk are enclosed in parentheses.

If the control device 1 on the other hand in accordance with FIG. 12 determines the maximum MAX, it modifies the sequences Sj which contain a smaller number of images Bk. This is because the control device 1 duplicates the last recorded image Bk for these sequences Sj, until these sequences Sj also contain the maximum MAX number of images Bk. This is indicated in FIG. 12 by the fact that the duplicates are enclosed in parentheses for the corresponding sequences Sj.

The subsequent processing of groups of images of the images Bk which now follows can alternatively be undertaken by means of the control device 1 or by means of an evaluation which is separate from this. It normally occurs in the form of a digital image subtraction. Thus it is possible for example within the framework of image acquisition, to record the first sequence S1 and only then to inject a contrast medium. The images Bk of the first sequence S1 in this case represent the reference images for the relevant image groups G1. Alternatively however formation of a differential image from immediately consecutive images. Bk of an image group G1 would be conceivable. Because of the fact that the image groups G1 each only contain images Bk which feature the same time offset in each case to the immediately preceding trigger pulse Ti, images Bk which at least essentially show the object 4 in the same phase can thus be subtracted from one another.

The differential images thus determined are then arranged chronologically again and this is done across groups of images. A user can thus be offered a greatly improved presentation of the contrast medium flow than would be possible without differential images.

Provided only sections of the images Bk are of importance, it is of course possible to select in one of the images Bk a corresponding section, known as the Region of Interest. This section is then transferred to the other images Bk. The image process described above is in this case only undertaken on the selected region.

Use of the inventive procedure thus enables a significantly less falsified and more artifact-free presentation to be achieved. The upgrading of existing medical imaging system required is essentially limited to a new control program 7, which e specially implements suitable triggering with the next trigger pulse Ti in each case.

1.-17. (canceled)

18. A method of operating a medical imaging system having a control device, an image recording device and a trigger device, the method comprising:
sequentially feeding a plurality of trigger pulses to the control device, by the trigger device, each trigger pulse corresponding to a predetermined phase angle of an object to be recorded, the predetermined phase angle being the same for all trigger pulses;
activating the image recording device by the control device, the image recording device triggered by each of at least part of the trigger pulses such that the image recording device records a sequence of images of the object, the recording of the sequence beginning at a start time relative to the respective trigger pulse and ending at a stop time relative to the respective trigger pulse such that each image of the sequence has a predetermined time offset relative to the respective trigger pulse, wherein the stop time is determined by the control device such that the stop time lies chronologically before a subsequent trigger impulse included in the part of the trigger pulses;
feeding the recorded sequences to the control device by the image recording device; and
storing the recorded sequences by the control device.

19. The method in accordance with claim 18, further comprising:
grouping the images of the recorded sequences into image groups so that such images having the same time offset are grouped into the same image group, by the control device; and
processing the images group-wise, by the control device.

20. The method in accordance with claim 18, further comprising:
determining an actual phase angle of the object by the trigger device; and
issuing one of the trigger pulses by the trigger device if the determined actual phase angle corresponds to a reference phase angle.

21. The method in accordance with claim 20, further comprising feeding a plurality of stimulation pulses to the object by a stimulation device.

22. The method in accordance with claim 18, further comprising:
feeding a plurality of stimulation pulses to the object by a stimulation device; and
deriving the trigger pulses from the stimulation pulses by the trigger device.

23. The method in accordance with claim 18, wherein the images of one of the sequences are spaced apart by the same time interval.

24. The method in accordance with claim 18, wherein the images of one of the sequences are spaced apart by different same time intervals.

25. The method in accordance with claim 18, wherein the part of trigger pulses activates the recording device only if a minimum waiting time has elapsed since recording a last image of a previous sequence.

26. The method in accordance with claim 18, wherein
each trigger pulse activates the recording device, and
the image recording device overwrites a last image of a previous sequence before feeding the recorded sequences to the control device.

27. The method in accordance with claim 18, wherein
each trigger pulse activates the recording device, and
the image recording device does not feed to the control device a first picture of at least one of the sequences.

28. The method in accordance with claim 18, wherein
each trigger pulses activates the recording device, and
the control device discards a first picture of each sequence.

29. The method in accordance with claim 18, further comprising:
inputting a trigger time interval to the control device, the trigger time interval corresponding to a chronological distance between the trigger pulses; and
determining the stop time based upon the trigger time interval and the respective start time, by the control device.

30. The method in accordance with claim 25, further comprising delaying
the start time by a delay time, the delay time equaling the minimum waiting time.

31. The method in accordance with claim 18, further comprising:
determining for each recorded sequence a number of images contained in the respective sequence, by the control device;
determining from the numbers of images a minimum number, by the control device; and
discarding such number of images of each sequence exceeding the minimum number, the discarded number of images including a last image and preceding images of the respective sequence.

32. The method in accordance with claim 18,, further comprising:
determining for each recorded sequence a number of images contained in the respective sequence, by the control device;
determining from the numbers of images a maximum number, by the control device; and
duplicating a last image of each sequence undershooting the maximum number such that the respective sequences have the maximum number of images after duplicating.

33. A computer program for operating a medical imaging system having a control device, an image recording device and a trigger device, the computer program having software modules programmed and configured to:
activate the trigger device to sequentially feed a plurality of trigger pulses to the control device, each trigger pulse corresponding to a predetermined phase angle of an object to be recorded, the predetermined phase angle being the same for all trigger pulses;
activate the image recording device using the control device, the image recording device triggered by each of at least part of the trigger pulses such that the image recording device records a sequence of images of the object, the recording of the sequence beginning at a start time relative to the respective trigger pulse and ending at a stop time relative to the respective trigger pulse such that each image of the sequence has a predetermined time offset relative to the respective trigger pulse, wherein the stop time is determined by the control device such that the stop time lies chronologically before a subsequent trigger impulse included in the part of the trigger pulses;
feed the recorded sequences to the control device using the image recording device; and
store the recorded sequences in the control device.

34. A medical imaging system, comprising:
a control device;
an image recording device;
a trigger device; and
a software program having software modules programmed and configured to:
activate the trigger device to sequentially feed a plurality of trigger pulses to the control device, each trigger pulse corresponding to a predetermined phase angle of an object to be recorded, the predetermined phase angle being the same for all trigger pulses;
activate the image recording device using the control device, the image recording device triggered by each of at least part of the trigger pulses such that the image recording device records a sequence of images of the object, the recording of the sequence beginning at a start time relative to the respective trigger pulse and ending at a stop time relative to the respective trigger pulse such that each image of the sequence has a predetermined time offset relative to the respective trigger pulse, wherein the stop time is determined by the control device such that the stop time lies chronologically before a subsequent trigger impulse included in the part of the trigger pulses;
feed the recorded sequences to the control device using the image recording device; and
store the recorded sequences in the control device.
